(19) United States
(12) Patent Application Publication
(10) Pub. No.: US 2025/0345582 A1
(43) Pub. Date: Nov. 13, 2025

(54) End Effectors for Instrument Delivery Devices

(71) Applicant: Becton, Dickinson and Company, Franklin Lakes, NJ (US)

(72) Inventors: Jonathan Karl Burkholz, Salt Lake City, UT (US); Curtis H. Blanchard, Herriman, UT (US)

(21) Appl. No.: 19/203,343

(22) Filed: May 9, 2025

Related U.S. Application Data
(60) Provisional application No. 63/645,391, filed on May 10, 2024.

(51) Int. Cl. A61M 39/10; A61M 39/00

(52) U.S. Cl. CPC A61M 39/1011; A61M 2039/0009; A61M 2039/1033; A61M 2039/1072

(57) ABSTRACT

Provided herein is a medical device including an instrument having a proximal end and a distal end, an introducer configured to moveably receive the instrument and having a housing having a proximal end, a distal end, and a sidewall therebetween defining an inner volume, and an end effector arranged at the distal end of the housing, the end effector configured to couple the introducer to an intravenous line and having a proboscis having a proximal end, a distal end, and a sidewall therebetween having a longitudinal axis and defining an interior that is configured to be in fluid communication with the inner volume of the introducer and the intravenous line, and at least one locking element configured to secure the end effector to the intravenous line.

### CROSS-REFERENCE TO RELATED APPLICATION

The present application claims priority to U.S. Provisional Patent Application No. 63/645,391, entitled “End Effectors for Instrument Delivery Devices” filed May 10, 2024, the entire disclosure of which is hereby incorporated by reference.

### BACKGROUND OF THE INVENTION

### Field of the Invention

The present disclosure relates generally to medical devices for use with intravenous (IV) catheters and, more specifically, to medical devices with features for improving performance in terms of accessing the vasculature.

### Description of Related Art

Instrument delivery devices, including those used for collecting blood, when used with indwelling IV catheters can include displaceable implements that are advanced beyond the tip of the indwelling catheter. Often, when the instrument is attached to an indwelling catheter, for example through a needle-free connector (NFC), there can be issues with maintaining a proper seal around the end effector of the instrument delivery device and maintaining a secure connection of the end effector to the NFC. In addition, given the geometries of the end effectors, moldability and manufacturability can be an issue. Accordingly, a need exists in the art for instrument delivery devices with improved manufacturability, and that provide a secure connection and robust performance of the instrument that is being delivered.

### SUMMARY OF THE INVENTION

Provided herein is a medical device including an instrument having a proximal end and a distal end, an introducer configured to moveably receive the instrument and having a housing having a proximal end, a distal end, and a sidewall therebetween defining an inner volume, and an end effector arranged at the distal end of the housing, the end effector configured to couple the introducer to an intravenous line and having a proboscis having a proximal end, a distal end, and a sidewall therebetween having a longitudinal axis of a varying outer diameter and defining an interior that is configured to be in fluid communication with the inner volume of the introducer and the intravenous line, and at least one locking element configured to secure the end effector to the intravenous line.

Also provided herein is a system, including a catheter adapter having a proximal end, a distal end, and a sidewall therebetween defining a fluid flow path, the catheter adapter having a needle at the distal end thereof and at least one connector at the proximal end thereof, and a medical device, including an instrument having a proximal end and a distal end, an introducer configured to moveably receive the instrument and having a housing having a proximal end, a distal end, and a sidewall therebetween defining an inner volume, and an end effector arranged at the distal end of the housing, the end effector configured to couple the introducer to the catheter adapter and including a proboscis having a proximal end, a distal end, and a sidewall therebetween having a longitudinal axis of a varying outer diameter and defining an interior that is configured to be in fluid communication with the inner volume of the introducer and the fluid flow path of the catheter adapter, and at least one locking element configured to secure the end effector to the at least one connector of the catheter adapter.

### BRIEF DESCRIPTION OF THE DRAWINGS

FIGS. 1A-1B show an instrument delivery device and end effector for the same;

FIGS. 2A-2B show a system (FIG. 2A) and needle-free connector (NFC) (FIG. 2B) to which end effectors described herein may be connected;

FIG. 3 shows an end effector for an instrument delivery device, according to non-limiting embodiments described herein;

FIG. 4 shows an end effector for an instrument delivery device, according to non-limiting embodiments described herein;

FIG. 5 shows an end effector for an instrument delivery device, according to non-limiting embodiments described herein;

FIG. 6 shows an end effector for an instrument delivery device, according to non-limiting embodiments described herein;

FIG. 7 shows an end effector for an instrument delivery device, according to non-limiting embodiments described herein;

FIGS. 8A-8C show an end effector for an instrument delivery device, according to non-limiting embodiments described herein; and

FIG. 9 shows an end effector for an instrument delivery device, according to non-limiting embodiments described herein.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, equivalents, variations, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

It should be understood that any numerical range recited herein is intended to include all values and sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value equal to or less than 10.

U.S. patent application Ser. No. 17/195,337 is incorporated herein by reference in its entirety.

Provided herein are devices and systems for delivering instruments through indwelling catheters, such as peripheral intravenous catheters (PIVCs). While certain devices (e.g., blood draw devices) are discussed below in terms of devices that may be used with PIVCs, and exemplified in the attached drawings, those of skill will appreciate that any number of different devices for introducing an instrument, including instruments ranging from tubes, probes, sensors (e.g., pressure sensors, pH sensors, lactate sensors, glucose sensors, and the like), wiring, fiber optics, guidewires, etc., may be used within the scope of the present disclosure. It is also to be understood that devices for delivering instruments as described herein may do so through a catheter adapter and/or a needle-free connector (NFC).

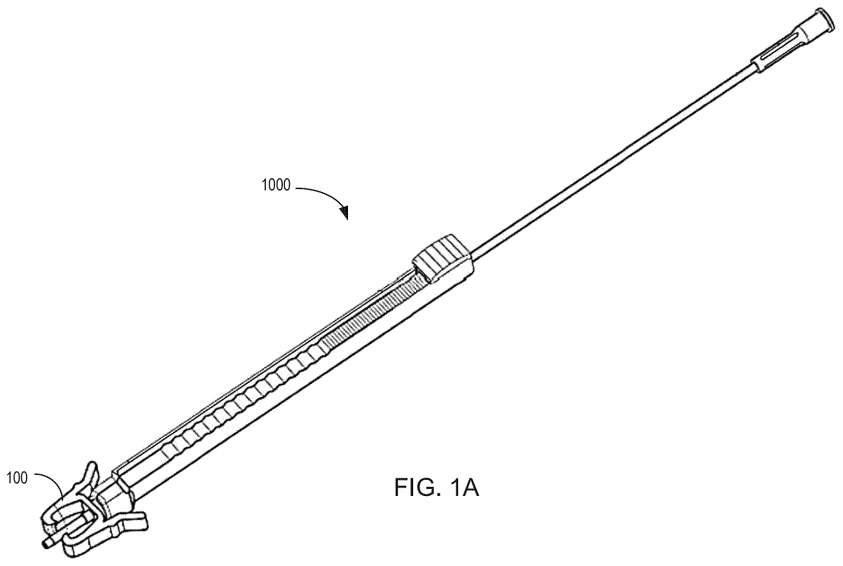

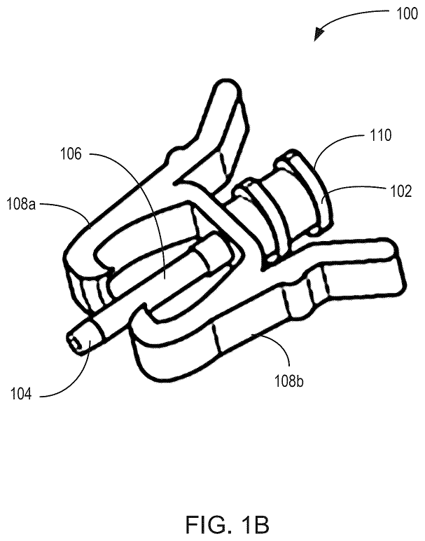

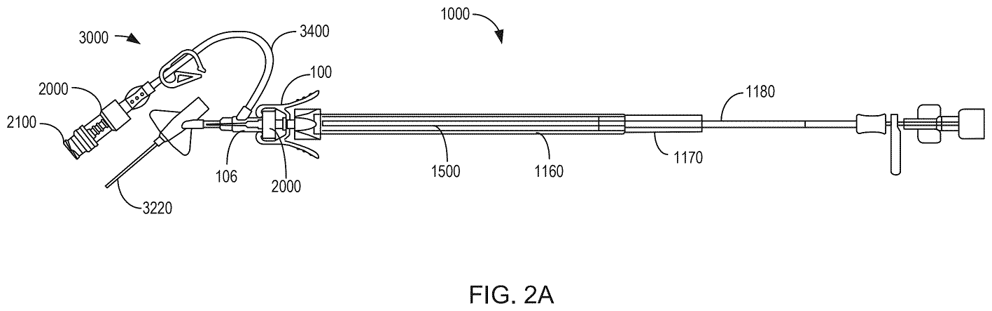

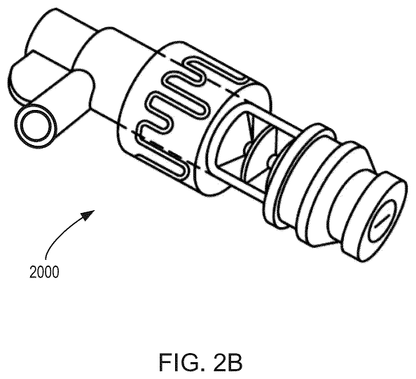

Referring now to FIGS. 1A-2B, shown are non-limiting embodiments of a medical device 1000 and a system including a medical device 1000 as described herein. System may include medical device 1000 and a catheter assembly 3000. Medical device 1000 may be an instrument delivery device in non-limiting embodiments. Suitable catheter assemblies 3000 for use with instrument delivery devices described herein are commercially available, for example from Becton, Dickinson and Company under the trade name Nexiva. Cather assembly 3000 may include a catheter adapter 3120, which may include a distal end and a proximal end. In some embodiments, the catheter adapter 3120 may include one or more ports to which a medical device, such as medical device 1000, may be coupled. In some embodiments, a port may be disposed at proximal end of catheter adapter 3120, or may be disposed between the distal end and the proximal end of catheter adapter 3120. In some embodiments, more than one port may be disposed between the distal end and the proximal end of catheter adapter 3120. In some embodiments, a catheter adapter 3120 may include at least one lumen extending through the distal end and the proximal end. Any lumen defined by a catheter may be sealed at proximal end of catheter adapter 3120.

In some non-limiting embodiments or aspects, the catheter assembly 3000 may include a catheter 3220 extending from the distal end. In some embodiments, the catheter 3220 may include a peripheral intravenous catheter, a midline catheter, or a peripherally-inserted central catheter. Catheter 3220 may be formed of any suitable material and may be of any useful length, as known to those of skill in the art. In some non-limiting embodiments or aspects, the catheter assembly 3000 may include any number of fluid conduits, for example fluid conduit 3400, extending from any port. Fluid conduits may be formed of any suitable material known to those of skill in the art, and may have a distal end and a proximal end, and may be coupled to any ports on catheter adapter 3000. In non-limiting embodiments, catheter adapter 3120 may have a NFC 2000 coupled thereto.

In some non-limiting embodiments or aspects, catheter assembly 3000 may include an extension set (integrated into or removably coupleable to catheter adapter 3120), including a fluid conduit such as fluid conduit 3400. Extension sets are known to those of skill in the art and are commercially available from, for example, Becton, Dickinson and Company. In some non-limiting embodiments or aspects, fluid conduit 3400 may include a luer connection at an end thereof. In some non-limiting embodiments or aspects, the extension set may include a clamp, to allow for occlusion of fluid conduit 3400. Clamps and fluid conduits may be formed of any suitable materials known to those of skill in the art.

Catheter assembly 3000 may include NFC 2000 and/or a fluid conduit 3400 as illustrated in FIG. 2A. Suitable NFCs 2000, such as shown in FIG. 2B, can include any split-septum connector and/or those with direct fluid path access. NFCs are known to those of skill in the art and are commercially available from, for example, Becton, Dickinson and Company under the trade names Q-SY TE, and SMARTSITE. While the non-limiting embodiments of FIGS. 1A-2B show a NFC arranged in a certain configuration, those of skill in the art will appreciate that suitable NFCs may also be arranged in other configurations. In non-limiting embodiments, NFCs include a septum (not shown), such as a self-healing septum. In non-limiting embodiments, the septum is a slit-type septum. As will be described below, medical device 1000 may be reversibly coupleable to NFC 2000 through end effector 100, and one or more portions of the medical device 1000 may pierce the septum and access the patient's vasculature through catheter 3220.

With continuing reference to FIGS. 1A-2B, medical device 1000 may include an outer housing 1160 having a proximal end and a distal end, and an inner housing. Throughout the illustrated embodiments, it will be clear to those of skill in the art that an intended effect of the instrument delivery devices described herein is that an instrument may be delivered (e.g., instrument, such as a fluid conduit 1500, may be moved from a first configuration, where instrument does not extend beyond distal end of outer housing 1160, to a second configuration, where instrument extends beyond distal end of outer housing 1160). In non-limiting embodiments, fluid conduit 1500 may be advanced from a first position, in which a distal end of fluid conduit 1500 is within medical device 1000, for example within outer housing 116 and/or lock end effector 100, and a second position, in which a distal end of fluid conduit 1500 is positioned distally of end effector 100 and, in embodiments in which medical device 1000 is coupled to catheter assembly 3000, optionally distally of catheter 3220. In non-limiting embodiments, medical device 1000 may include one or more inner housings 1170, 1180, to allow for compression and/or telescoping of housings, such that a total length of the housing(s) of medical device 1000 is less than when the instrument has not been delivered.

Turning to FIG. 1B, shown is an end effector 100 for a medical device 1000. End effector 100 may be a device for coupling medical device 100 to a vascular access device (VAD), such as catheter assembly 3000. End effector 100 may have a proximal end 102, a distal end 104, and a sidewall therebetween defining an interior 110 that may be fluidly coupled to at least one lumen of catheter adapter 3200, fluid conduit 3400, NFC 2000, and/or to interior of housing 1160 of medical device 1000, such as fluid conduit 1500. In non-limiting embodiments, end effector 100 includes proboscis 106, which may pierce a septum of an NFC, such as NFC 2000. In non-limiting embodiments, end effector 100 may also include a plurality of arms 108a, 108b, to secure end effector 100 to catheter assembly 3000, for example to NFC 2000.

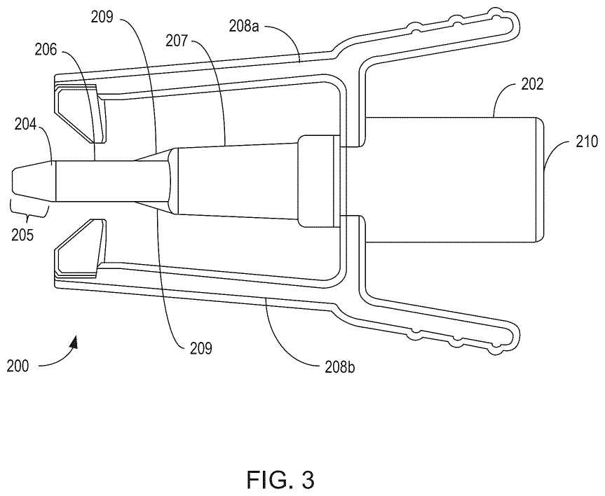

Turning to FIGS. 3-7, shown are non-limiting embodiments of end effector, in particular, configurations of proboscis and/or luer of an end effector. With regard to FIG. 3, shown is end effector 200, which may have a proximal end 202, a distal end 204 (which may be blunted and/or rounded), and a sidewall therebetween defining an interior 210 that may be fluidly coupled to at least one lumen of catheter adapter 3200, fluid conduit 3400, NFC 2000, and/or to interior of housing 1160 of medical device 1000, such as fluid conduit 1500. In non-limiting embodiments, end effector 200 includes a proboscis 205, 206, 207 which may pierce a septum of an NFC, such as NFC 2000. In the illustrated non-limiting embodiment, proboscis may vary in outer diameter along a longitudinal axis thereof, between a first portion 205, second portion 206, and a third portion 207. In non-limiting embodiments, first portion 205 may have a smaller outer diameter than second portion 206, which may have a smaller outer diameter than third portion 207. In non-limiting embodiments, first portion 205 may itself have a varying outer diameter, and may have a smaller outer diameter at a distal end thereof (at or near distal end 204 of proboscis) and a larger outer diameter at a proximal end thereof (at or near second portion 206). In non-limiting embodiments, end effector 200 may also include a plurality of arms 208a, 208b, to secure end effector 200 to catheter assembly 3000, for example to NFC 2000. As shown in the non-limiting embodiment depicted in FIG. 3, proboscis may include a transition region 209 between second portion 206 and third portion 207. In the non-limiting embodiment depicted in FIG. 3, transition region 209 is a ramped surface, providing a region of substantially constant slope between second portion 206 and third portion 207. While not illustrated, those of skill in the art will appreciate that a slope of transition region 209 may vary along a longitudinal axis thereof, and/or transition region 209 may have another configuration, such as one or more steps. Without wishing to be bound by the theory, it is believed that the blunted and/or rounded distal end 204 of proboscis 205, 206, 207, and/or transition region 209 provides increased sealing pressure and/or reduces damage to the septum of an NFC, such as NFC 2000.

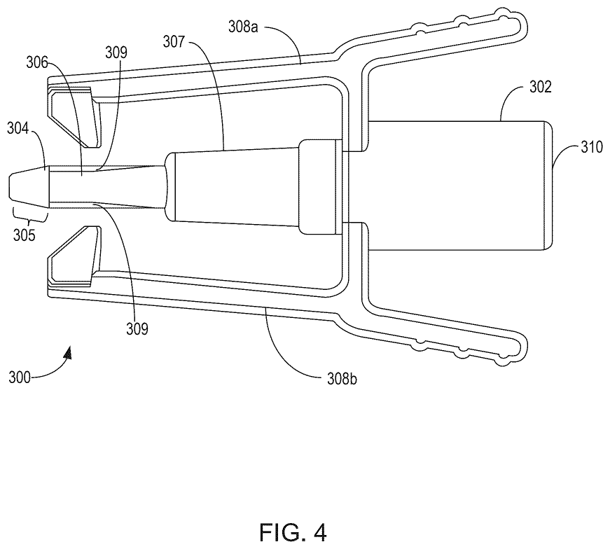

Turning to FIG. 4, shown is end effector 300, which may have a proximal end 302, a distal end 304 (which may be blunted and/or rounded), and a sidewall therebetween defining an interior 310 that may be fluidly coupled to at least one lumen of catheter adapter 3200, fluid conduit 3400, NFC 2000, and/or to interior of housing 1160 of medical device 1000, such as fluid conduit 1500. In non-limiting embodiments, end effector 300 includes a proboscis 305, 306, 307 which may pierce a septum of an NFC, such as NFC 2000. In the illustrated non-limiting embodiment, proboscis may vary in outer diameter along a longitudinal axis thereof, between a first portion 305, second portion 306, and a third portion 307. In non-limiting embodiments, first portion 305 may have a smaller outer diameter than second portion 306, which may have a smaller outer diameter than third portion 307. In non-limiting embodiments, first portion 305 may itself have a varying outer diameter, and may have a smaller outer diameter at a distal end thereof (at or near distal end 304 of proboscis) and a larger outer diameter at a proximal end thereof (at or near second portion 306). In non-limiting embodiments, end effector 300 may also include a plurality of arms 308a, 308b, to secure end effector 200 to catheter assembly 3000, for example to NFC 2000. As shown in the non-limiting embodiment depicted in FIG. 4, one or more portions of proboscis, such as portion 306, may include one or more features 309 to increase sealing pressure and/or reduce damage to the septum of an NFC, such as NFC 2000. In non-limiting embodiments, one or more features 309 may include one or more indentations in proboscis, for example in first portion 307. In non-limiting embodiments, one or more indentations may be concave in nature. Without wishing to be bound by the theory, it is believed that the blunted and/or rounded distal end 304 of proboscis 305, 306, 307, transition region, and/or one or more features 309 provides increased sealing pressure and/or reduces damage to the septum of an NFC, such as NFC 2000.

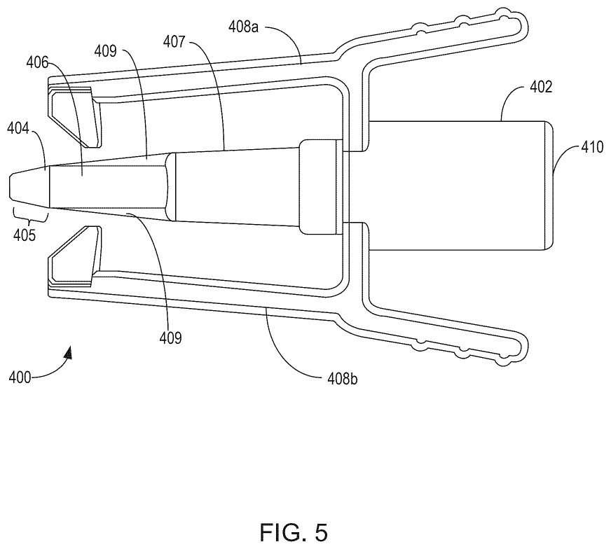

With reference to FIG. 5, shown is end effector 400, which may have a proximal end 402, a distal end 404 (which may be blunted and/or rounded), and a sidewall therebetween defining an interior 410 that may be fluidly coupled to at least one lumen of catheter adapter 3200, fluid conduit 3400, NFC 2000, and/or to interior of housing 1160 of medical device 1000, such as fluid conduit 1500. In non-limiting embodiments, end effector 400 includes a proboscis 405, 406, 407 which may pierce a septum of an NFC, such as NFC 2000. In the illustrated non-limiting embodiment, proboscis may vary in outer diameter along a longitudinal axis thereof, between a first portion 405, second portion 406, and a third portion 407. In non-limiting embodiments, first portion 405 may have a smaller outer diameter than second portion 406, which may have a smaller outer diameter than third portion 407. In non-limiting embodiments, first portion 405 may itself have a varying outer diameter, and may have a smaller outer diameter at a distal end thereof (at or near distal end 404 of proboscis) and a larger outer diameter at a proximal end thereof (at or near second portion 406). In non-limiting embodiments, end effector 400 may also include a plurality of arms 408a, 408b, to secure end effector 400 to catheter assembly 3000, for example to NFC 2000. As shown in the non-limiting embodiment depicted in FIG. 3, proboscis may include a transition region 409 between second portion 406 and third portion 407. In the non-limiting embodiment depicted in FIG. 5, transition region 409 is a ramped surface, providing a region of substantially constant slope between second portion 406 and third portion 407. It may be appreciated that the embodiment depicted in FIG. 5 differs from that of FIG. 3 at least in the longitudinal distance that the transition region 409 occupies. While not illustrated, those of skill in the art will appreciate that a slope of transition region 409 may vary along a longitudinal axis thereof. Without wishing to be bound by the theory, it is believed that the blunted and/or rounded distal end 404 of proboscis 405, 406, 407 and/or transition region 409 provides increased sealing pressure and/or reduces damage to the septum of an NFC, such as NFC 2000.

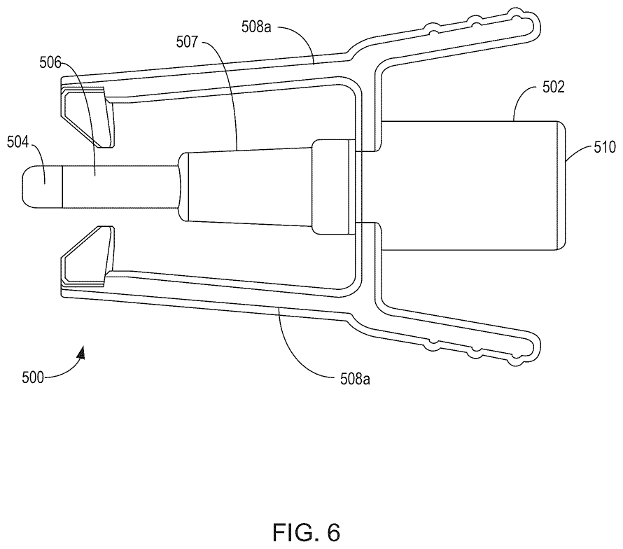

With reference to FIG. 6, shown is end effector 500, which may have a proximal end 502, a distal end 504, and a sidewall therebetween defining an interior 510 that may be fluidly coupled to at least one lumen of catheter adapter 3200, fluid conduit 3400, NFC 2000, and/or to interior of housing 1160 of medical device 1000, such as fluid conduit 1500. In non-limiting embodiments, end effector 500 includes a proboscis 506, 507 which may pierce a septum of an NFC, such as NFC 2000. In the illustrated non-limiting embodiment, proboscis may vary in outer diameter along a longitudinal axis thereof, between a first portion 506, and a second portion 507. In non-limiting embodiments, first portion 506 may have a smaller outer diameter than second portion 407. In non-limiting embodiments, for example as shown here, distal end 504 of proboscis 506, 507 may be rounded. In non-limiting embodiments, end effector 500 may also include a plurality of arms 508a, 508b, to secure end effector 500 to catheter assembly 3000, for example to NFC 2000. As shown in the non-limiting embodiment depicted in FIG. 5, proboscis may include a transition region between first portion 506 and second portion 507. Without wishing to be bound by the theory, it is believed that the rounded distal end 504 of proboscis 506, 507 and/or the transition region provides increased sealing pressure and/or reduces damage to the septum of an NFC, such as NFC 2000.

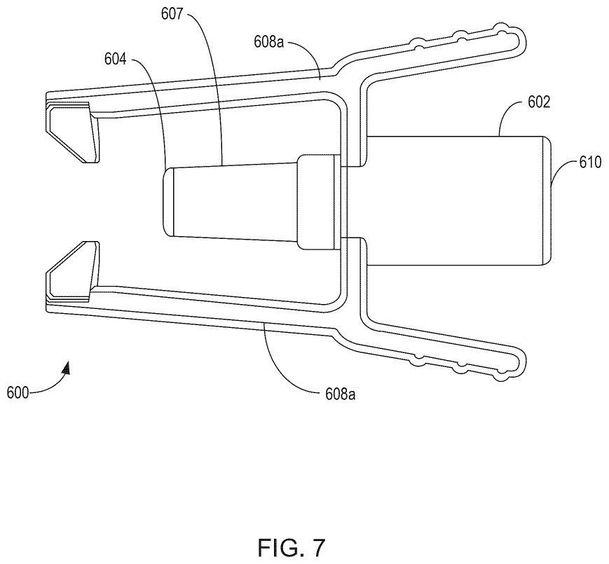

With reference to FIG. 7, shown is end effector 600, which may have a proximal end 602, a distal end 604 (that may be blunted and/or rounded), and a sidewall therebetween defining an interior 610 that may be fluidly coupled to at least one lumen of catheter adapter 3200, fluid conduit 3400, NFC 2000, and/or to interior of housing 1160 of medical device 1000, such as fluid conduit 1500. In non-limiting embodiments, end effector 600 includes a proboscis 607 which may pierce a septum of an NFC, such as NFC 2000. In the illustrated non-limiting embodiment, proboscis may vary in outer diameter only at distal end 604. In non-limiting embodiments, end effector 600 may also include a plurality of arms 608a, 608b, to secure end effector 600 to catheter assembly 3000, for example to NFC 2000. Without wishing to be bound by the theory, it is believed that the distal end 604 of proboscis 607 provides increased sealing pressure and/or reduces damage to the septum of an NFC, such as NFC 2000.

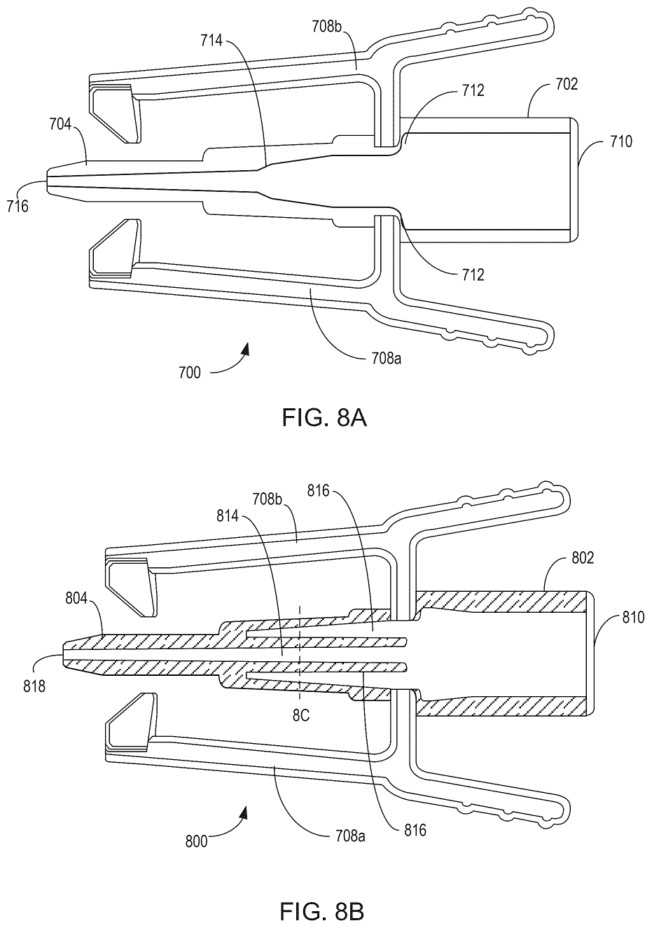

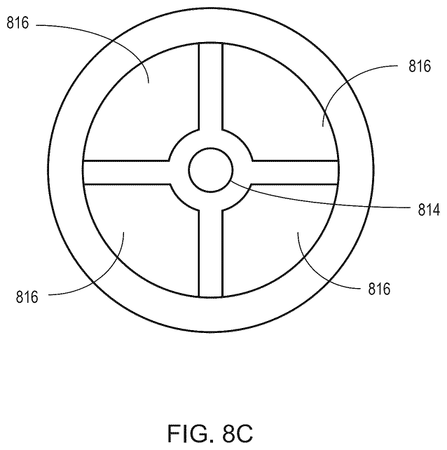

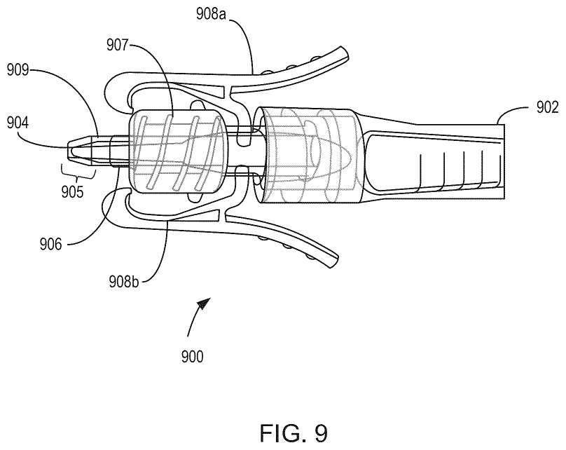

Turning to FIGS. 8A-8C, shown are non-limiting embodiments of an end effector. With regard to FIG. 8A, shown is end effector 700, which may have a proximal end 702, a distal end 704 (that may be blunted and/or rounded), and a sidewall therebetween defining an interior that may be fluidly coupled to at least one lumen of catheter adapter 3200, fluid conduit 3400, NFC 2000, and/or to interior of housing 1160 of medical device 1000, such as fluid conduit 1500. End effector 700 includes a proboscis which may pierce a septum of an NFC, such as NFC 2000. In the illustrated non-limiting embodiment, proboscis may vary in outer diameter as shown in FIGS. 3-7, and as described above. In non-limiting embodiments, end effector 700 may also include a plurality of arms 708a, 708b, to secure end effector 700 to catheter assembly 3000, for example to NFC 2000. In the illustrated non-limiting embodiment, interior may have a varying inner diameter along a longitudinal axis of end effector 700. For example, interior may have a first portion 710, second portion 714, and third portion 716, each of which may have a different inner diameter. For example, first portion 710 may have a larger diameter than second portion 714, which may have a larger inner diameter than third portion 716. In non-limiting embodiments, the inner diameter of the interior may have a constant slope between one or more of the portions 710, 714, 716. In non-limiting embodiments, the inner diameter of the interior may have a slope that differs between one or more of the portions 710, 714, 716. In non-limiting embodiments, at a transition between one or more of first portion 710 and second portion 714, and/or second portion 714 and third portion 716, one or more step regions 712 may be included.

With regard to FIGS. 8B-8C, shown is end effector 800, which may have a proximal end 802, a distal end 804 (that may be blunted and/or rounded), and a sidewall therebetween defining an interior that may be fluidly coupled to at least one lumen of catheter adapter 3200, fluid conduit 3400, NFC 2000, and/or to interior of housing 1160 of medical device 1000, such as fluid conduit 1500. End effector 800 includes a proboscis which may pierce a septum of an NFC, such as NFC 2000. In the illustrated non-limiting embodiment, proboscis may vary in outer diameter as shown in FIGS. 3-7, and as described above. In non-limiting embodiments, end effector 800 may also include a plurality of arms 808a, 808b, to secure end effector 800 to catheter assembly 3000, for example to NFC 2000. In the illustrated non-limiting embodiment, interior may have a varying inner diameter along a longitudinal axis of end effector 800. For example, interior may have a first portion 810, second portion 814, and third portion 816, each of which may have a different inner diameter. For example, first portion 810 may have a larger diameter than second portion 814, which may have a larger inner diameter than third portion 816. In non-limiting embodiments, the inner diameter of the interior may have a constant slope between one or more of the portions 810, 814, 818. In non-limiting embodiments, the inner diameter of the interior may have a slope that differs between one or more of the portions 810, 814, 818. In non-limiting embodiments, at a transition between one or more of first portion 810 and second portion 814, and/or second portion 814 and third portion 818, one or more step regions may be included.

As shown more clearly in FIG. 8C, in non-limiting embodiments, interior of end effector 800 may include one or more channels 816 that extend along the longitudinal axis of end effector 800, but do not extend the full length of the longitudinal axis (for example, as shown in FIG. 8B). In non-limiting embodiments, a plurality of channels 816 surrounds portion 814, through which an instrument of medical device 1000 may pass. Without wishing to be bound by the theory, the internal configurations of an end effector shown in FIGS. 8A-8C are believed to provide improved moldability and manufacturability.

Turning to FIG. 9, shown is a non-limiting embodiment of end effector 900, which may have a proximal end 902, a distal end 904 (which may be blunted and/or rounded), and a sidewall therebetween defining an interior 910 that may be fluidly coupled to at least one lumen of catheter adapter 3200, fluid conduit 3400, NFC 2000, and/or to interior of housing 1160 of medical device 1000, such as fluid conduit 1500. In non-limiting embodiments, end effector 900 includes a proboscis 905, 906, which may pierce a septum of an NFC, such as NFC 2000. Proboscis 905, 906 may have any configuration disclosed herein, including varying diameters and/or transition regions. In the illustrated non-limiting embodiment, proboscis may vary in outer diameter along a longitudinal axis thereof, between a first portion 905 and second portion 906. In non-limiting embodiments, first portion 905 may have a smaller outer diameter than second portion 906. In non-limiting embodiments, first portion 905 may itself have a varying outer diameter, and may have a smaller outer diameter at a distal end thereof (at or near distal end 904 of proboscis) and a larger outer diameter at a proximal end thereof (at or near second portion 906). In non-limiting embodiments, end effector 900 may also include a plurality of arms 908a, 908b, to secure end effector 900 to catheter assembly 3000, for example to NFC 2000. As shown in the non-limiting embodiment depicted in FIG. 3, proboscis may include a transition region 909 between first portion 905 and second portion 906. Without wishing to be bound by the theory, it is believed that the blunted and/or rounded distal end 904 of proboscis 905, 906, and/or transition region 909, provides increased sealing pressure and/or reduces damage to the septum of an NFC, such as NFC 2000. With continuing reference to FIG. 9, in non-limiting embodiments end effector 900 includes a luer component, to aid in securing end effector 900 to catheter assembly 3000, for example an NFC such as NFC 2000. In non-limiting embodiments the luer component 907 is a threaded luer component.

Although the present disclosure has been described in detail for the purpose of illustration based on what is currently considered to be the most practical and preferred embodiments or aspects, it is to be understood that such detail is solely for that purpose and that the present disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment may be combined with one or more features of any other embodiment.

The invention claimed is:

1. A medical device, comprising:
an instrument having a proximal end and a distal end;
an introducer configured to moveably receive the instrument and having a housing having a proximal end, a distal end, and a sidewall therebetween defining an inner volume; and
an end effector arranged at the distal end of the housing, the end effector configured to couple the introducer to an intravenous line and comprising:
a proboscis having a proximal end, a distal end, and a sidewall therebetween having a longitudinal axis of a varying outer diameter and defining an interior that is configured to be in fluid communication with the inner volume of the introducer and the intravenous line; and
at least one locking element configured to secure the end effector to the intravenous line.

2. The medical device of claim 1, wherein the at least one locking element comprises a luer connector.

3. The medical device of claim 2, wherein the luer connector comprises a threaded luer.

4. The medical device of claim 1, wherein the at least one locking element comprises a plurality of arms configured to grasp the intravenous line.

5. The medical device of claim 1, wherein the distal end of the proboscis is blunted.

6. The medical device of claim 1, wherein the distal end of the proboscis is rounded.

7. The medical device of claim 1, wherein the sidewall of the proboscis comprises one or more first portions having a first outer diameter and one or more second portions having a second outer diameter.

8. The medical device of claim 7, wherein the first outer diameter is larger than the second outer diameter.

9. The medical device of claim 7, wherein the one or more first portions are arranged proximally of the one or more second portions.

10. The medical device of claim 7, wherein the sidewall of the proboscis comprises one or more transition regions between the one or more first portions and the one or more second portions.

11. The medical device of claim 10, wherein the one or more transition regions comprise one or more steps.

12. The medical device of claim 10, wherein the one or more transition regions comprise a ramped surface.

13. The medical device of claim 1, wherein the sidewall of the proboscis comprises an outer diameter that varies along the longitudinal axis between the proximal end of the proboscis and the distal end of the proboscis in at least three regions.

14. The medical device of claim 1, wherein the sidewall of the proboscis comprises one or more indentations.

15. The medical device of claim 14, wherein the one or more indentations are concave indentations.

16. A system, comprising:
a catheter adapter comprising a proximal end, a distal end, and a sidewall therebetween defining a fluid flow path, the catheter adapter comprising a needle at the distal end thereof and at least one connector at the proximal end thereof; and
a medical device, comprising:
an instrument having a proximal end and a distal end;
an introducer configured to moveably receive the instrument and having a housing having a proximal end, a distal end, and a sidewall therebetween defining an inner volume; and
an end effector arranged at the distal end of the housing, the end effector configured to couple the introducer to the catheter adapter and comprising:
a proboscis having a proximal end, a distal end, and a sidewall therebetween having a longitudinal axis of a varying outer diameter and defining an interior that is configured to be in fluid communication with the inner volume of the introducer and the fluid flow path of the catheter adapter; and
at least one locking element configured to secure the end effector to the at least one connector of the catheter adapter.

17. The system of claim 16, wherein the connector is a needle-free connector.

18. The system of claim 16, wherein the at least one locking element comprises a luer connector.

19. The system of claim 18, wherein the luer connector comprises a threaded luer.

20. The system of claim 16, wherein the at least one locking element comprises a plurality of arms configured to grasp at least one connector.
